(19) United States
(12) Patent Application Publication
(10) Pub. No.: US 2005/0181031 A1
(43) Pub. Date: Aug. 18, 2005

(54) Solifenacin transdermal preparation and method for enhancing transdermal permeation thereof

(76) Inventors: Katsumi Saito, Yaizu-shi (JP); Masataka Katsuma, Yaizu-shi (JP)

Correspondence Address: KILYK & BOWERSOX, P.L.L.C., 53 A EAST LEE STREET, WARRENTON, VA, 20186, US

(21) Appl. No.: 11/061,858

(22) Filed: Feb. 18, 2005

Related U.S. Application Data
(60) Provisional application No. 60/545,623, filed on Feb. 18, 2004.

(51) Int. Cl. A61K 31/4745; A61L 15/16; A61K 9/70

(52) U.S. Cl. 424/448; 514/305

(57) ABSTRACT

The transdermal permeable property of solifenacin or a salt thereof as a biologically active substance, is remarkably improved using a fatty acid ester, a terpene or the like as a selected transdermal permeable promoter. The difference in effect exceeded expectations by producing an enhancement in permeability that represents an increase of several hundredfold at most, and several tenfold for practical application, enabling the provision of extremely useful means for preventive and therapeutic agents for urologic diseases or respiratory diseases that use solifenacin.

This application claims benefit under 35 U.S.C. §119(e) of prior U.S. Provisional Patent Application No. 60/545,623, filed Feb. 18, 2004, the entire contents of which are incorporated herein by reference.

### TECHNICAL FIELD

The present invention relates to a transdermal preparation having as a principal component (+)-(1S,3′R)-quinuclidin-3′-yl 1-phenyl-1,2,3,4-tetrahydroisoquinoline-2-carboxylate (hereunder, referred to simply as “solifenacin”) or a salt thereof, and a method for enhancing transdermal permeation of the same.

### BACKGROUND ART

A transdermal preparation is a preparation that delivers a biologically active substance into the body via the skin, and recent years have seen increasing activity in the research and development of transdermal preparations. For patients who have difficulty swallowing, such as elderly people, administration of orally administered drugs such as tablets and capsules is difficult. Further, administration by injection requires the patient to visit a hospital or clinic, and in the case of a chronic disease, daily administration places a large burden on the patient. In contrast, administration of a transdermal preparation can be carried out independently by the patient by pasting or applying the preparation onto the skin, and in addition to facilitating self-administration, it also has an advantage that the patient can immediately stop administration of the drug in a case where side effects are evident. Thus, because administration of transdermal preparations is simple and convenient for a variety of patients including the elderly and children, they are garnering attention as formulations that can be expected to enhance patient compliance.

However, the original function of the structure of the skin is to protect the individual from changes in the external environment, and in particular, the stratum corneum that is located on the outermost surface of the skin fulfills this function. Accordingly, the skin has a function to restrict the infiltration of substances, and thus the transdermal permeation of biologically active substances is also extremely restricted. Although there are some biologically active substances such as nitroglycerin and nicotine that have a small molecular weight and low melting point and which are known to have favorable transdermal permeability, in general the transdermal permeability of biologically active substances is extremely low, and it is extremely difficult to deliver an effective amount of drug into the body via the skin. Consequently, to solve this problem, a large number of studies have been reported relating to substances that enhance the transdermal permeability of biologically active substances. For example, the transdermal permeation enhancers described hereunder are known.

The known transdermal permeation enhancers include diethyl sebacate (International Patent Publication No. WO 92/19271), lauric acid diethanolamide (Japanese Patent Laid-Open No. 11-335281), N-methyl-2-pyrrolidone (Japanese Patent Laid-Open No. 8-113533), lauroyl macrogol-6 glyceride (International Patent Publication No. 95/28932, U.S. Pat. No. 5,503,843), propylene glycol monolaurate (Japanese Patent Laid-Open No. 8-40937, U.S. Pat. No. 5,059,426, U.S. Pat. No. 5,053,227, U.S. Pat. No. 4,973,468, U.S. Pat. No. 5,006,342, U.S. Pat. No. 4,906,463, U.S. Pat. No. 5,006,342), triacetin (Japanese Patent Laid-Open No. 2001-39865, U.S. Pat. No. 5,601,839: transdermal permeation enhancer of basic drugs having pKa of approximately 8.0 or more), isopropyl myristate and isopropyl palmitate (Japanese Patent Laid-Open No. 2001-131089, Japanese Patent Laid-Open No. 8-225448), and 1-menthol (Japanese Patent Laid-Open No. 2000-119195).

Solifenacin is a quinuclidine derivative that is a potent and selective muscarinic M₃ receptor antagonist (Japanese Patent No.3014457, U.S. Pat. No. 6,017,927). It is expected that the pharmacological action thereof will be useful as a preventive and therapeutic agent for urologic diseases (overactive bladder, irritable bowel syndrome, nervous pollakisuria, neurogenic bladder, nocturnal enuresis, detrusor instability, cystospasm, chronic cystitis and the like) or respiratory diseases (chronic obstructive respiratory disease, chronic bronchitis, asthma, rhinitis and the like).

However, a solifenacin transdermal preparation and method for enhancing the transdermal permeation thereof are still unknown.

### DISCLOSURE OF THE INVENTION

An object of the present invention is to provide a solifenacin transdermal preparation and a method for enhancing the transdermal permeation thereof.

The present inventors focused on the fact that in many cases the target patients for prevention and treatment of urologic diseases or respiratory diseases which are susceptible to treatment with solifenacin are elderly individuals, and set about developing a transdermal preparation from the viewpoint of convenience. However, our experiments revealed that it is difficult to deliver solifenacin itself into the body via the skin in an effective amount.

Therefore, in order to supply solifenacin or a salt thereof into the body transdermally, we examined various means for promoting the transdermal permeability of the drug.

In consideration of species dependent differences in transdermal permeability, we estimated that what is needed, for effective clinical results for solifenacin is a skin flux of at least 40 μkg/cm²/h in the in vitro skin permeability test through excised hairless mice skin. We evaluated various popular agents for promoting the transdermal permeability of the drug. However, they could not achieve the skin flux that we estimate to be necessary for effective clinical results.

But we found that a certain kind of transdermal permeation enhancer can achieve the desired effect specifically, to thereby complete the present invention. That is, in the present invention we discovered that by employing a means that utilizes solifenacin or a salt thereof as a biologically active substance, and utilizes a fatty acid ester, a terpene or the like as a selected transdermal permeation enhancer independently or in combination of two or more kinds thereof, the transdermal permeability of solifenacin or a salt thereof is remarkably enhanced, and completed this invention. The transdermal permeation enhancer selected in this invention achieves and exceeds the level of skin flux that we estimate to be necessary for effective clinical results, even if such selected enhancer is used independently. Further, we found that we can achieve the desired clinical result for solifenacin or a salt thereof.

More specifically, the present invention relates to the following.

1. A solifenacin transdermal preparation, comprising:
1) solifenacin (chemical name: (+)-(1S,3′R)-quinuclidin-3′-yl 1-phenyl-1,2,3,4-tetrahydroisoquinoline-2-carboxylate) or a salt thereof; and
2) at least one transdermal permeation enhancer selected from the group consisting of a fatty acid ester and a terpene.

2. The transdermal preparation according to the above 1, wherein the fatty acid ester is an ester formed between a carboxylic acid having 6 to 22 carbon atoms and an alkyl alcohol having 1 to 12 carbon atoms.

3. The transdermal preparation according to the above 2, wherein the fatty acid ester is at least one member selected from the group consisting of isopropyl myristate, isopropyl palmitate and butyl myristate.

4. The transdermal preparation according to the above 1, wherein the terpene is at least one member selected from the group consisting of 1-menthol, limonene and cineole.

5. The transdermal preparation according to the above 4, which is further mixed with water to adjust a pH to 5.0-8.0.

6. The transdermal preparation according to any one of the above 1 to 5, wherein the transdermal preparation further contains at least one adhesive selected from an acrylic type, rubber type, and silicone type.

7. A method for enhancing a transdermal permeation property of a solifenacin preparation containing solifenacin (chemical name: (+)-(1S,3′R)-quinuclidin-3′-yl 1-phenyl-1,2,3,4-tetrahydroisoquinoline-2-carboxylate) or a salt thereof, comprising at least one process selected from the group consisting of the following 1) and 2):
1) providing an admixture system with a fatty acid ester; and
2) providing an admixture system with a terpene.

8. A method for enhancing a transdermal permeation property of a solifenacin or a salt thereof, comprising putting a transdermal preparation containing solifenacin (chemical name: (+)-(1S,3′R)-quinuclidin-3′-yl 1-phenyl-1,2,3,4-tetrahydroisoquinoline-2-carboxylate) or a salt thereof in a mixture system with water and a terpene which is adjusted a pH to approximately 5.0-8.0.

### DETAILED DESCRIPTION OF THE INVENTION

Hereunder, the transdermal preparation containing solifenacin or a salt thereof of the present invention is described in further detail.

A drug used in the present invention is at least one kind selected from the group consisting of solifenacin and salts thereof. Examples of salts of solifenacin include the inorganic salts of hydrochloric acid, sulfuric acid, hydrobromic acid and so on, or the acid addition salts by organic acids such as acetic acid, succinic acid, oxalic acid, maleic acid, fumaric acid, citric acid, and lactic acid. In the present invention, examples of particularly preferable drugs include solifenacin free base and solifenacin succinate.

In the present invention, an enhancement or improvement of the transdermal permeability means increasing in the transdermal permeability of solifenacin. For example, when the level of skin flux of solifenacin is over 40 μg/cm²/h in the in vitro permeability test through excised hairless mice skin by using Franz-type diffusion cells, it is considered that the transdermal permeability is enhanced or improved. However, this level of skin flux is just an estimate based on what is necessary for effective clinical results and is not intended to be limiting.

By using a selected transdermal permeation enhancer, the present invention makes it possible to achieve a greater transdermal permeation effect for solifenacin. In the present invention, one or more kinds selected from the group consisting of a fatty acid ester and a terpene can be used as a selected transdermal permeation enhancer.

Examples of the fatty acid ester used in the transdermal preparation of the present invention include an ester formed between an aliphatic monocarboxylic acid having 6 to 22 carbon atoms, preferably 10 to 20, more preferably 12 to 18, and an alkyl alcohol having 1 to 12 carbon atoms, preferably 2 to 8, more preferably 3 to 6, and more specifically isopropyl myristate, isopropyl palmitate and butyl myristate are preferable. As more preferable examples, isopropyl myristate, and isopropyl palmitate may be mentioned. The fatty acid ester can achieve a promoting effect for both a solifenacin salt and a solifenacin free base.

Examples of the terpene used in the transdermal preparation of the present invention include 1-menthol, limonene, cineole and menthone. As a more preferable example, 1-menthol may be mentioned. Further, peppermint oil, orange oil, turpentine oil, eucalyptus oil and the like that contain these terpenes as principal components can be used. Terpenes can achieve a promoting effect for a solifenacin salt in particular, and the mixture preparation with water is preferable, and a preparation that has been subjected to pH adjustment is further preferable. More specifically, it is desirable to adjust the preparation to a pH of approximately 5.0-8.0, preferably approximately 6.0-8.0, and most preferably approximately 7.0-8.0. When the pH value is less than 5.0, solifenacin is dissociated at so high a concentration in the solution that an effective dose of transdermal permeation is difficult. When the pH value is greater than 8.0, deposition of solifenacin free base is a concern. The term “a mixture preparation with water” refers to a preparation in which solifenacin or a salt thereof is mixed with water to give a lotion, ointment, gel, or a patch by use of a moistened non-woven fabric.

The form of a preparation by the present invention that contains solifenacin or a salt thereof as a principal component and is improved in transdermal permeability is not particularly limited as long as it is a pharmaceutically acceptable form. Specific examples thereof include preparations such as a monolithic-type patch, a poultice, a gel, a cream, a gelled cream, a plaster, a reservoir-type patch, a liniment, an aerosol, an ointment, a lotion, a spray and an emulsion that can be applied to the skin to express the pharmacological effect of the principal component locally or systemically. Methods for preparing the various formulations of the transdermal preparation of the present invention are not particularly limited, and methods that are conventionally used as methods for preparing external preparations can be used. For example, detailed preparation methods for various kinds of formulations described from page 3 onwards of Japanese Patent No. 2852816 (WO 94/26309) can be applied.

Where necessary, the transdermal preparation of the present invention may contain a pharmaceutically acceptable excipient [for example, an oil soluble base such as vaseline, solid paraffin, olive oil, sesame oil, cottonseed oil, liquid paraffin, lanolin, a higher fatty acid alcohol (for example, cetanol, stearyl alcohol, or oleyl alcohol), a higher fatty acid (for example, myristic acid, palmitic acid, stearic acid, or oleic acid), a wax (for example, white beeswax, yellow bees wax, or spermaceti wax), a lipid (for example, soybean lecithin, dipalmitoylphosphatidylcholine, distearoylphosphatidylcholine), or silicone oil, and a water soluble base such as a macrogol (for example, macrogol 400, macrogol 600, macrogol 1000, macrogol 1500, macrogol 4000, or macrogol 6000), an alkanediol having 2-5 carbon atoms (for example, glycerin), an alkanetriol having 2-5 carbon atoms (for example, propylene glycol or 1,3-butylene glycol), polyvinyl alcohol (PVA), polyacrylic acid (PAA), sodium polyacrylate, carboxyvinyl polymer or a highly water absorptive resin (for example, a block polymer of PVA and PAA, such as Sumica gel SP-510, Sumitomo Chemical Co., Ltd.) may be mentioned, and these may be used independently or in combination of two or more kinds thereof]; a gelling agent (for example, carboxyvinyl polymer, hydroxyethyl cellulose, hydroxypropylcellulose, carboxymethylcellulose, sodium alginate, polyvinyl alcohol, dried aluminium hydroxide gel or agar); a pH regulator (for example, an inorganic acid such as hydrochloric acid or sulfuric acid or an organic acid such as acetic acid, succinic acid or maleic acid); an alkali (such as sodium hydroxide, potassium hydroxide or triethanolamine); a stabilizer; a solubilizing auxiliary agent (such as α-cyclodextrin, β-cyclodextrin or γ-cyclodextrin); a solubilizer [for example, an ethylene glycol (monoethylene glycol, diethylene glycol, triethylene glycol, or diethylene glycol monoethyl ether), glycerin, propylene glycol, 1,3-butylene glycol, polyethylene glycol, polypropylene glycol, methyl alcohol, ethyl alcohol, propanol, isopropanol, butanol, t-butanol, hexanol, octanol or so on]; an antioxidant [such as ascorbic acid, stearate ester, sodium ascorbate, a tocopherol (1-form, d-form, or d1-form of α-tocopherol, β-tocopherol, γ-tocopherol and δ-tocopherol) and ester derivatives of these, nordihydroguaiaretic acid, dibutyl hydroxytoluene, a tert-butylhydroquinone gallate (an ester such as ethyl, propyl or isoamyl), 1-oxo-3-methyl-4-isopropyl benzene or so on]; a surfactant [for example, polyoxyethylene sorbitan fatty acid ester, polyoxyethylene sorbitol fatty acid ester, polyoxyethylene fatty acid ester, polyoxyethylene higher alcohol ether, polyoxyethylene alkyl aryl ether, a polyoxyethylene castor oil derivative, a nonionic surfactant such as a nonionic block polymer surfactant (Pluronic L-62, L-64 or F-68), or an ionic surfactant such as sodium lauryl sulfate (SLS)]; a buffering agent (for example, a buffer solution such as phosphate, acetate, carbonate, citrate or so on); a thickening agent (for example, curdlan, agar, mucin, gelatin, pectin, carageenan, chitin, chitosan, locust bean gum, gum tragacanth, xanthan gum, pullulan, sucralfate or so on); an adhesive [for example, an acrylic pressure sensitive adhesive (for example, a copolymer comprising at least two members of the group consisting of 2-ethylhexyl acrylate, vinyl acetate, ethyl acrylate, methacrylate, methoxyethyl acrylate, and copolymer of at least two type of acrylic acid (e.g. Duro-Tak 2287, manufactured by National Starch & Chemical Co., Ltd.)], a silicone type adhesive (for example, polysiloxane), a rubber type adhesive [for example, natural and synthetic rubbers: polyisobutylene (PIB), neoprene, polybutadiene, polyisoprene or so on), polyurethane, styrene-isoprene-styrene block copolymer (SIS), styrene-butadiene-styrene block copolymer (SBS) and the like may be mentioned, and these may be used independently or in combinations of two or more]; an adhesion-strengthening agent (for example, a natural resin such as a rosin resin or a terpene resin, a petroleum resin typically represented by C5 or C9 resins, a synthetic resin such as coumarone-indene resin or so on); and an antiseptic (such as benzoic acid, sodium benzoate, ethyl parahydroxybenzoate, propyl parahydroxybenzoate, butyl parahydroxybenzoate or so on).

The amount of solifenacin or a salt thereof contained in the transdermal preparation of the present invention is not particularly limited as long as the amount is a therapeutically effective amount or a prophylactically effective amount. Preferably, the amount is 0.5-85 wt % relative to the entire transdermal preparation of the present invention, and more preferably 1-70 wt %. A further preferable amount of the drug is 1-60 wt %, and a still further preferable amount is 1-50 wt %.

The amount of a transdermal permeation enhancer is not particularly limited, as long as it is an amount that can cause transdermal permeation of a therapeutically effective amount or prophylactically effective amount of solifenacin or a salt thereof. In the case of a fatty acid ester or a terpene, a preferable amount is 0.1-50 wt %, and more preferably is 0.2-40 wt %. Further preferably, the amount is 0.5-30 wt %. If the amount is less than 0.1 wt %, it is difficult to cause transdermal permeation of an effective dose of solifenacin or a salt thereof. Further, if the mixing proportion is 50 wt % or more, there is a concern that skin irritation may occur. In this connection, in the case of solifenacin free base, isopropyl myristate and isopropyl palmitate may be exemplified as preferred transdermal permeation enhancers, and a mixing proportion thereof is preferably 5-25 wt %, more preferably 5-20 wt %, and particularly preferably 5-15wt %. And a mixing proportion in the case of using terpene is preferably 0.1-20 wt %, more preferably 0.2-15 wt %, and particularly preferably 0.5-10 wt %. If the amount is less than 0.1 wt %, it is difficult to cause transdermal permeation of an effective dose of solifenacin or a salt thereof. Further, if the mixing proportion is 20 wt % or more, there is a concern that skin irritation may occur.

### EXAMPLES

The present invention is described more specifically below by means of examples, however the scope of the present invention is not limited by the following examples.

Further, the amount of each kind of transdermal permeation enhancer can be decided upon considering an amount of exposure that causes skin irritation or a normally used amount.

(A) Improvement of transdermal permeation by use of fatty acid esters or terpenes

### Example 1

### Solifenacin Succinate Transdermal Preparation (Transcutol/IPM)

0.1 g of solifenacin succinate was dissolved in 3.9 g of diethylene glycol monoethyl ether (Transcutol P®, Gattefosse Corp.), 1 g of isopropyl myristate (IPM-EX®, Nikko Chemical Co., Ltd.) was then mixed therein, and the mixture was stirred to obtain a solifenacin transdermal preparation.

### Example 2

### Solifenacin Free Base Transdermal Preparation (Transcutol/IPM)

0.1 g of solifenacin free base was dissolved in 3.9 g of diethylene glycol monoethyl ether (Transcutol P®, Gattefosse Corp.), 1 g of isopropyl myristate (IPM-EX®, Nikko Chemical Co., Ltd.) was then mixed therein, and the mixture was stirred to obtain a solifenacin transdermal preparation.

### Example 3

### Solifenacin Free Base Transdermal Preparation (Transcutol/IPP)

0.1 g of solifenacin free base was dissolved in 3.9 g of diethylene glycol monoethyl ether (Transcutol P®, Gattefosse Corp.), 1 g of isopropyl palmitate (IPP®, Nikko Chemical Co., Ltd.) was then mixed therein, and the mixture was stirred to obtain a solifenacin transdermal preparation.

### Example 4

### Solifenacin Succinate Transdermal Preparation (Transcutol/1-menthol)

0.1 g of solifenacin succinate was dissolved in 2.8 g of diethylene glycol monoethyl ether (Transcutol P®, Gattefosse Corp.), and 0.1 g of 1-menthol (Nacalai Tesque Inc.) was further added thereto and dissolved. Subsequently, 1.75 g of phosphate buffer solution (pH 7.4) and 0.25 g of IN-sodium hydroxide solution were mixed therein, and the mixture was stirred to obtain a solifenacin transdermal preparation.

### Example 5

### Solifenacin Free Base Transdermal Preparation (Transcutol/1-menthol)

0.1 g of solifenacin free base was dissolved in 2.8 g of diethylene glycol monoethyl ether (Transcutol P®, Gattefosse Corp.), and 0.1 g of 1-menthol (Nacalai Tesque Inc.) was further added thereto and dissolved. Subsequently, 2 g of purified water was mixed therein, and the mixture was stirred to obtain a solifenacin transdermal preparation.

### Example 6

### Solifenacin Succinate Transdermal Preparation (Transcutol/ R+limonene)

0.1 g of solifenacin succinate was dissolved in 2.8 g of diethylene glycol monoethyl ether (Transcutol P®, Gattefosse Corp.), and 0.1 g of R+limonene (Kanto Chemical Co., Ltd.) was further added thereto and dissolved. Subsequently, 1.75 g of phosphate buffer solution (pH 7.4) and 0.25 g of 1N-sodium hydroxide solution were mixed therein, and the mixture was stirred to obtain a solifenacin transdermal preparation.

### Example 7

### Solifenacin Free Base Transdermal Preparation (Transcutol/IPM/1-menthol)

0.1 g of solifenacin free base was dissolved in 3.8 g of diethylene glycol monoethyl ether (Transcutol P®, Gattefosse Corp.), and 1 g of isopropyl myristate (IPM-EX®, Nikko Chemical Co., Ltd.) and 0.1 g of 1-menthol (Nacalai Tesque Inc.) was further added thereto, dissolved and mixed therein, and the mixture was stirred to obtain a solifenacin transdermal preparation.

### Example 8

### (Monolithic Patch Using Acrylic Adhesive/IPM)

1 g of isopropyl myristate (IPM-EX®, Nikko Chemical Co., Ltd.) was mixed in ethyl acetate solution containing 7 g of acrylic pressure sensitive adhesive polymer (Duro-Tak 2287, National Starch & Chemical Co., Ltd.), and 2 g of solifenacin free base was dissolved therein. The mixture was coated onto a release liner to obtain a thickness after drying of 100 μm, and then dried to form a adhesive layer. Subsequently, the adhesive layer was stuck on a backing layer (12 μm thick polyester film) to obtain a transdermal preparation according to the present invention.

### Comparative Example 1

### Solifenacin Succinate Transdermal Preparation (Verifying Permeation of the Preparation Consisting of Only Purified Water and Solifenacin Succinate)

0.1 g of solifenacin succinate was dissolved in 4.9 g of purified water to obtain a solifenacin transdermal preparation.

### Comparative Example 2

### Solifenacin Free Base Transdermal Preparation (Transcutol; no Enhancer)

0.1 g of solifenacin free base was dissolved in 4.9 g of diethylene glycol monoethyl ether (Transcutol P®, Gattefosse Corp.) to obtain a solifenacin transdermal preparation.

### Comparative Example 3

### Solifenacin Succinate Transdermal Preparation (Transcutol/Lauric Acid)

0.1 g of solifenacin succinate was dissolved in 4.4 g of diethylene glycol monoethyl ether (Transcutol P®, Gattefosse Corp.), 0.5 g of lauric acid (Kanto Kagaku Chemical Co., Ltd.) was then mixed therein, and the mixture was stirred to obtain a solifenacin transdermal preparation.

### Comparative Example 4

### Solifenacin Succinate Transdermal Preparation (Transcutol/NMP)

0.1 g of solifenacin succinate was dissolved in 4.8 g of diethylene glycol monoethyl ether (Transcutol P®, Gattefosse Corp.), 0.1 g of N-methyl-2-pyrrolidone (Pharmasolve®, ISP Japan Ltd.) was then mixed therein, and the mixture was stirred to obtain a solifenacin transdermal preparation.

### Comparative Example 5

### Solifenacin Succinate Transdermal Preparation (Transcutol/Labrafil)

0.1 g of solifenacin succinate was dissolved in 4.65 g of diethylene glycol monoethyl ether (Transcutol P®, Gattefosse Corp.), 0.25 g of lauroyl macrogol-6 glyceride (Labrafil M 2130CS®, Gattefosse Corp.) was then mixed therein, and the mixture was stirred to obtain a solifenacin transdermal preparation.

### Comparative Example 6

### Solifenacin Free Base Transdermal Preparation (Transcutol/PGML)

0.1 g of solifenacin free base was dissolved in 3.9 g of diethylene glycol monoethyl ether (Transcutol P®, Gattefosse Corp.), 1 g of propylene glycol monolaurate (Lauroglycol 90®, Gattefosse Corp.) was then mixed therein, and the mixture was stirred to obtain a solifenacin transdermal preparation.

### Comparative Example 7

### Solifenacin Free Base Transdermal Preparation (Transcutol/Triacetin)

0.1 g of solifenacin free base was dissolved in 3.9 g of diethylene glycol monoethyl ether (Transcutol P®, Gattefosse Corp.), 1 g of triacetin (Daicel Chemical Industries, Ltd.) was then mixed therein, and the mixture was stirred to obtain a solifenacin transdermal preparation.

### Experimental Example 1

### In Vitro Transdermal Permeability Test

The transdermal permeability of the preparations described in Examples 1 to 6,and 8 and Comparative Examples 1 to 7 was tested using hairless mice. Skin of female hairless mice (5 weeks old; purchased from Charles River Japan) was detached and set in Franz-type diffusion cells. 0.5 ml of each of the preparations prepared in the examples and comparative examples was administered on the acceptor side of the respective diffusion cells, and the amount of solifenacin that permeated to the receptor side with time was determined. The transdermal permeation rate of each transdermal preparation was calculated based on the drug amount that permeated across the skin for 24 hours. An isotonic phosphate buffer solution (pH 7.4) was used as the receptor solution. The cell temperature was set at 32° C.

TABLE 1
Result of in vitro transdermal permeability test

| | Skin Flux |
|---|---|
| | (μg/cm²/h) |
| Example 1 | 78.6 |
| Example 2 | 189.2 |
| Example 3 | 159.1 |
| Example 4 | 314.3 |
| Example 5 | 98.3 |
| Example 6 | 274.1 |
| Example 8 | 51.7 |
| Comparative Example 1 | 4.0 |
| Comparative Example 2 | 0.6 |
| Comparative Example 3 | 2.3 |
| Comparative Example 4 | 1.1 |
| Comparative Example 5 | 3.7 |
| Comparative Example 6 | 16.2 |
| Comparative Example 7 | 0.4 |

Comparative Example 1 shows the permeability of solifenacin succinate solution, Comparative Example 2 shows the permeability of solifenacin free base in a vehicle without a permeation enhancer, and Comparative Examples 3 to 7 show the results when known transdermal permeation enhancers were mixed with solifenacin free base or a succinate thereof.

Results and Discussion

As shown in Table 1, remarkably high transdermal permeability was observed for the solifenacin transdermal preparations obtained in Examples 1 to 6 and 8 in comparison to the preparations obtained in the comparative examples. The difference in effect ranged from several tenfold to several hundredfold, achieving an enhancement in transdermal permeability that exceeded expectations.

(B) Transdermal Preparations (Patch, Gel, Ointment, Spray)

### Example 9

### (Monolithic Patch Using Acrylic Adhesive)

1 g of isopropyl palmitate (IPP-EX®, Nikko Chemical Co., Ltd.) was mixed in ethyl acetate solution containing 7 g of acrylic pressure sensitive adhesive polymer (Duro-Tak 2287, National Starch & Chemical Co., Ltd.), and 2 g of solifenacin free base was dissolved therein. The mixture was coated onto a release liner to obtain a thickness after drying of 100 μm, and then dried to form a adhesive layer. Subsequently, the adhesive layer was stuck on a backing layer (12-μm thick polyester film) to obtain a transdermal preparation according to the present invention.

### Example 10

### (Monolithic Patch Using PIB Rubber Adhesive)

1.4 g of hydrocarbon tackifier (ARKON P-100, Arakawa Chemical Industries, Ltd.) was added to 5.6 g of polyisobutylene adhesive polymer (Himol 6H, Shin Nippon Petrochemicals Co., Ltd.), and toluene was further added thereto and uniformly dissolved. To this solution was added 1 g of isopropyl myristate (IPM-EX®, Nikko Chemical Co., Ltd.), and 2 g of solifenacin free base was then dissolved therein. The mixture was coated onto a release liner to obtain a thickness after drying of 100 μm, and then dried to form a grease layer. Subsequently, the adhesive layer was stuck on a backing layer(12-μm thick polyester film) to obtain a transdermal preparation according to the present invention.

### Example 11

### (Monolithic Patch Using SIS Rubber Adhesive)

1.4 g of hydrocarbon tackifier (ARKON P-100, Arakawa Chemical Industries, Ltd.) was added to 5.6 g of styrene-isoprene-styrene block copolymer (SIS) (Califlex TR1101,Shellchemicals Co., Ltd.), and toluene was further added thereto and uniformly dissolved. To this solution was added 1 g of isopropyl myristate (IPM-EX®, Nikko Chemical Co., Ltd.), and 2 g of solifenacin free base was then dissolved therein. The mixture was coated onto a release liner to obtain a thickness after drying of 100 μm, and then dried to form a grease layer. Subsequently, the adhesive layer was stuck on a backing layer(12-μm thick polyester film) to obtain a transdermal preparation according to the present invention.

### Example 12

### (Monolithic Patch Using Silicone Type Adhesive)

Heptane solution containing 7 g of Silicone type adhesive polymer (BIO-PSA R7-4201,Dow coming Asia Co., Ltd.)was prepared. To this solution was added 1 g of isopropyl myristate (IPM-EX®, Nikko Chemical Co., Ltd.), and 2 g of solifenacin free base was then dissolved therein. The mixture was coated onto a release liner to obtain a thickness after drying of 100 μm, and then dried to form a grease layer. Subsequently, the adhesive layer was stuck on a backing layer(12-μm thick polyester film) to obtain a transdermal preparation according to the present invention.

### Example 13

### (Gel)

1 g of carbopol was added to 30 g of water and expanded, after which 0.5 g of triethanolamine was added to cause gelation. Meanwhile, 2 g of solifenacin succinate was dissolved in 16.5 g of water, the pH was adjusted to 7.4 with sodium hydroxide, 50 g of Transcutol was mixed therein, and 2 g of 1-menthol was dissolved in the mixture. The thus-obtained two mixtures were mixed to form a gel.

### Example 14

### (Ointment)

To 2 g of solifenacin free base were added 80 g of white vaseline, 10 g of isopropyl myristate, 3 g of spermaceti wax, 2 g of polyoxyethylene lauryl ether sodium phosphate, and 0.05 g of butyl parahydroxybenzoate, the mixture was heated to maintain at 80° C., and after all the components formed a clear solution, the solution was mixed uniformly with a homomixer. Thereafter, the solution was returned to room temperature while stirring to obtain an ointment.

### Example 15

### (Spray)

After dissolving 2 g of solifenacin succinate in 78 g of diethylene glycol monoethyl ether, 20 g of isopropyl myristate was mixed therein, and the mixture was stirred to obtain a solifenacin solution. A pressure storage spray container (15 mL capacity; spray amount: approximately 0.05 mL) was filled with 10 mL of the solution to obtain a spray.

### INDUSTRIAL APPLICABILITY

As described in the foregoing, a transdermal administration method of the present invention that employs as a biologically active substance solifenacin or a salt thereof achieves excellent transdermal permeability of solifenacin. The difference in effect exceeded expectations by producing an enhancement in permeability that represents an increase of several hundredfold at most, and several tenfold for practical application, enabling the provision of extremely useful means for preventive and therapeutic agents for urologic diseases or respiratory diseases that use solifenacin.

1. A solifenacin transdermal preparation, comprising:
1) solifenacin (chemical name: (+)-(1S,3′R)-quinuclidin-3′-yl 1-phenyl-1,2,3,4-tetrahydroisoquinoline-2-carboxylate) or a salt thereof; and
2) at least one transdermal permeation enhancer selected from the group consisting of a fatty acid ester and a terpene.

2. The transdermal preparation according to claim 1, wherein the fatty acid ester is an ester formed between a carboxylic acid having 6 to 22 carbon atoms and an alkyl alcohol having 1 to 12 carbon atoms.

3. The transdermal preparation according to claim 2, wherein the fatty acid ester is at least one member selected from the group consisting of isopropyl myristate, isopropyl palmitate and butyl myristate.

4. The transdermal preparation according to claim 1, wherein the terpene is one or more members selected from 1-menthol, limonene and cineole.

5. The transdermal preparation according to claim 4, which is further mixed with water to adjust a pH to 5.0-8.0.

6. The transdermal preparation according to claim 1, further containing at least one adhesive selected from an acrylic type, rubber type, and silicone type.

7. A method for improving transdermal permeation property of a transdermal preparation containing solifenacin (chemical name: (+)-(1S,3′R)-quinuclidin-3′-yl 1-phenyl-1,2,3,4-tetrahydroisoquinoline-2-carboxylate) or a salt thereof, comprising at least one kind of process selected from the group consisting of the following 1) and 2):
1) Providing an admixture system with a fatty acid ester; and
2) Providing an admixture system with a terpene.

8. A method for improving a transdermal permeation property of a solifenacin preparation in a form of admixture that comprises solifenacin (chemical name: (+)-(1S,3′R)-quinuclidin-3′-yl 1-phenyl-1,2,3,4-tetrahydroisoquinoline-2-carboxylate) or a salt thereof, a terpene and water, the method comprising adjusting a pH of the admixture to approximately 5.0-8.0.

9. The transdermal preparation according to claim 2, further containing at least one adhesive selected from an acrylic type, rubber type, and silicone type.

10. The transdermal preparation according to claim 3, further containing at least one adhesive selected from an acrylic type, rubber type, and silicone type.

11. The transdermal preparation according to claim 4, further containing at least one adhesive selected from an acrylic type, rubber type, and silicone type.

12. The transdermal preparation according to claim 5, further containing at least one adhesive selected from an acrylic type, rubber type, and silicone type.
